(19) United States
(12) Patent Application Publication
(10) Pub. No.: US 2005/0026852 A1
(43) Pub. Date: Feb. 3, 2005

(54) Method of augmenting the antitumor activity of anticancer agents

(76) Inventors: Youcef M. Rustum, Amherst, NY (US); Shousong Cao, East Amherst, NY (US); Farukh Durrani, Snyder, NY (US)

Correspondence Address: HODGSON RUSS LLP, ONE M & T PLAZA, SUITE 2000, BUFFALO, NY, 14203-2391, US

(21) Appl. No.: 10/844,800

(22) Filed: May 13, 2004

Related U.S. Application Data
(60) Provisional application No. 60/471,183, filed on May 13, 2003.

(51) Int. Cl. A61K 31/704; A61K 31/513; A61K 31/198; A61K 31/337

(52) U.S. Cl. 514/34; 514/449; 514/109; 514/561; 514/492; 514/49; 514/269

(57) ABSTRACT

A method for augmenting the antitumor activity of anti-cancer agents is provided. The method comprises administering to an individual an anti-cancer agent and a selenium compound. The selenium compounds may be administered before, during or after administration of the anti-cancer agent.

This application claims priority to U.S. provisional application No. 60/471,183 filed on May 13, 2003, the disclosure of which is incorporated herein by reference.

### FIELD OF THE INVENTION

This invention relates generally to the field of cancer therapy and more particularly to a method for augmenting the antitumor activity of chemotherapeutic agents.

### DESCRIPTION OF RELATED ART

Chemotherapy is now a recognized and widely used modality of cancer treatment. Depending upon the type of cancer, chemotherapy is often the primary course of treatment. For example, chemotherapy is widely used either alone or in combination with other treatments such as radiation treatment for a variety of cancers including cancer of the ovary, testis, breast, bladder, colon, head and neck as well as leukemia, lymphomas, sarcomas, melanomas, myelomas and others.

Chemotherapeutic agents are broadly classified into a number of groups. The majority of anticancer drugs act as cytotoxic drugs. The classification of these drugs into groups is mechanism based. While chemotherapeutic agents have proven extremely useful in the treatment of cancer, nearly all of them are associated with significant toxic effects because of their potential to kill cancerous as well as healthy cells. The toxicity associated with anticancer drugs often forces discontinuation of treatment which may negatively impact the prognosis of patient's condition and clinical outcome and result in compromising the quality of life.

In the field of cancer therapy there is an ongoing need to identify new chemotherapeutic agents or to increase the potency of existing agents. While some recent in vitro studies have attempted to address the issue of toxicity of anticancer agents by selenium compounds (Steifel et al., 1999, WO 99/64018; Chen et al., 1986, J. Nutrition, 116(12):2453-2465; Dobric et al., 1998, J. Environ. Pathol. Toxicol Oncol., 17:291-299, the effects of selenium on the antitumor activity of the chemotherapeutic agents, if any, are unknown.

### SUMMARY OF THE INVENTION

In the present invention it was observed that administration of selenium compounds augments the antitumor activity of anticancer agents. Data is presented for in vivo studies in xenograft bearing animals.

Accordingly, the present invention discloses a method for augmenting the antitumor activity of anticancer agents. The method comprises administering to an individual having a tumor, an anti-tumor agent and a selenium compound. The selenium compounds may be administered before, during or after administration of the anti-cancer agent. In one embodiment, the selenium compound is administered prior to chemotherapy and may be continued during and after the chemotherapy.

### BRIEF DESCRIPTION OF THE FIGURES

FIG. 1 is a representation of the effect of selenium on the antitumor activity of irinotecan (CPT-11) in nude mice bearing HCT-8 colon xenografts irinotecan was administered by i.v. push once a week for 4 weeks and methylselenocysteine (MSC) by oral route (p.o.) daily for 28 days with the first dose administered 7 days prior to the administration of irinotecan.

FIG. 2 is a representation of the effect of selenium compounds on the antitumor activity of irinotecan in colon carcinoma and squamous cell carcinoma of the head and neck xenograft tumors for irinotecan alone or in combination with MSC (0.2 mg/mouse/day for 42 days) was administered for 4 weeks. Irinotecan was administered by intravenous (i.v.) push. Irinotecan was started on day 7 after the initiation of the MSC administration. *, ** and *** indicates toxic doses of 50%, 100% and 20% lethality. Animals which survived toxic doses of irinotecan were used to calculate tumor effect.

FIG. 3 is a representation of the effectiveness of MSC and SLC in enhancing the antitumor activity of xenograft bearing A253 and FaDu tumors. Irinotecan was used at a concentration of 100 mg/kg.

FIG. 4 is another representation of the effectiveness of two selenium compounds on the antitumor activity of irinotecan. Irinotecan was used at a concentration of 200 mg/kg.

FIG. 5 is a representation of the effect of MSC on the antitumor activity of drugs cisplatin, taxol, cyclophosphamide and doxorubicin on A253 and FaDu tumors for control (●), drug alone (▴) and drug plus MSC (▪).

FIG. 6 is a representation of the effect of selenium on the median tumor weight in rats bearing advanced Ward colorectal carcinoma when treated with control or oxaliplatin alone or in combination with MSC. Oxaliplatin was administered by a single i.v. injection and MSC (0.75 mg/rat/day) by p.o. daily for 21 days with the first dose given 14 days before oxaliplatin treatment. Each group had 8 rats from 2 independent experiments.

FIG. 7 is a representation of the effect of selenium on the antitumor activity of oxaliplatin in rats bearing advanced colorectal carcinoma. Data is presented for oxaliplatin alone and for oxaliplatin in combination with MSC. Oxaliplatin was administered by a single i.v. injection and MSC 0.75 mg/rat/day p.o. daily for 21 days and the first dose started 14 days before oxaliplatin treatment. Each group had 8 rats from 2 independent experiments.

FIG. 8 is a representation of the effect of MSC on the antitumor activity of dxorubicin and oxaliplatin against human A253 and FaDu head and neck xenograft for control, drug alone and for drug in combination with MSC. Each group had 10 mice from 2 independent experiments.

FIG. 9 is another representation of the effect of MSC on the antitumor activity of doxorubicin or oxaliplatin on A253 and FaDu tumors. Doxorubicin was administered by a single i.v. injection and oxaliplatin by i.v. push weekly for 4 weeks. MSC (0.2 mg/mouse/day) was administered by p.o. daily for 14 days with doxorubicin and 28 days with oxaliplatin, and the first dose was started 7 days before chemotherapy. Each group had 10 mice from 2 independent experiments.

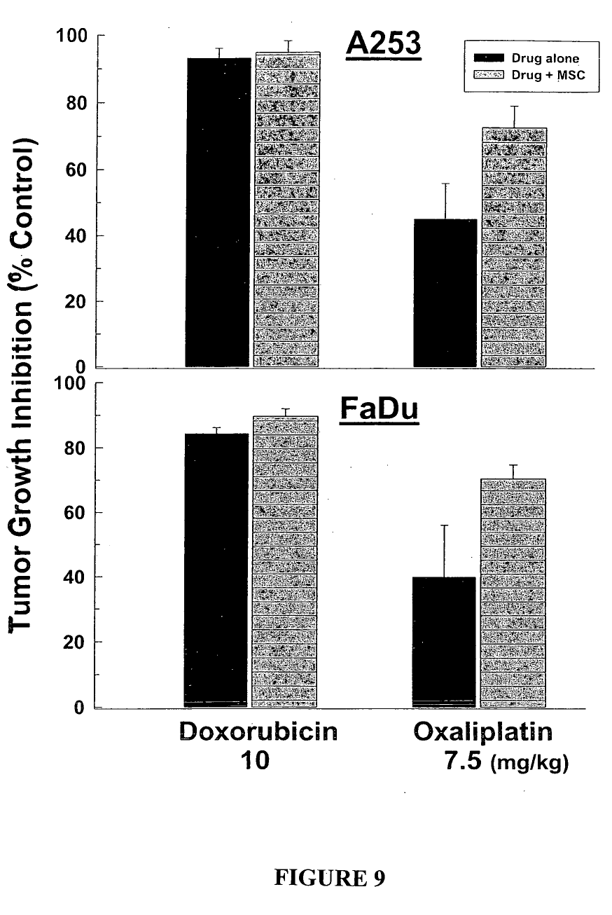

FIG. 10 is a representation of the effect of MSC on the antitumor activity of taxotere against human A253 and FaDu head and neck xenografts for control, drug alone and for drug in combination with MSC.

FIG. 11 is a representation of the effect of selenium on mean body weight of nude mice as a function of time when administered alone or in combination with taxotere. Taxotere was administered by a single intravenous injection and MSC was administered at 0.2/mg/mouse/day by p.o. daily for 14 days with the first dose started 7 days before taxotere treatment.

FIG. 12 is a representation of the effect of selenium on taxotere induced toxicity in rats measured as percent of survivors. Taxotere alone or in combination with MSC was administered at the indicated doses. MSC was administered by p.o. daily for 14 days with the first dose started 7 days before taxotere treatment

### DETAILED DESCRIPTION OF THE INVENTION

The term “therapeutic dose” as used herein means the dosage of a therapeutic agent that is acceptable for use clinically with respect to its toxicity without the co-administration of selenium compounds.

The term “cure” as used herein means the complete disappearance of a tumor. A tumor is considered to have completely disappeared when it is undetectable by palpation.

The present invention discloses a method for augmenting the antitumor activity of anticancer agents. The method comprises administering to an individual, in need of such a treatment, one or more anticancer agents and one or more selenium compounds. The selenium compounds may be administered before, during or after administration of the anticancer agent. By combining chemotherapy with the administration of selenium compounds, the antitumor toxicity of the chemotherapeutic agent can be increased.

This invention is useful for augmenting the anti-tumor activity of anticancer agents including fluoropyrimidines, pyrimidine nucleosides, purines, platinum analogues, antroacyclines, podophyllotoxins, camptothecins, hormones and hormone analogues, enzymes, proteins and antibodies, vinca alkaloids, taxanes. The anti-cancer agents for the present invention generally fall into one or more of the following functional categories: antihormones, antifolates, antimicrotubule agents, alkylating agents, antimetabolites, antibiotics, topoisomerase inhibitors and antivirals.

Selenium compounds useful for the present invention can be from either organic or inorganic forms. It is preferable to use selenium from organic forms since these are known to be less toxic. Examples of useful selenium compounds from organic forms include methylselenocysteine (MSC) and seleno-L-methionine (SLM). The doses of selenium compounds are in the range of about 200 μg/person to about 3.6 mg/person and maybe administered daily for 1 year or longer. It has been reported that up to 800 μg/patient is generally considered to be safe without associated toxicity.

The present invention comprises the steps of combining chemotherapy with the administration of selenium. One or more chemotherapeutic agents may be used accordingly to the criteria well known in the art of cancer chemotherapeutics. The dosage and administrative regimens of the chemotherapeutics are well within the purview of those skilled in the art. Selenium administration can be initiated before the start of chemotherapy, during chemotherapy or after cessation of chemotherapy. If initiated before the start of chemotherapy, selenium administration can be continued during the chemotherapy and after cessation of chemotherapy. Similarly, if initiated during chemotherapy, selenium administration can continue after cessation of chemotherapy.

While the present method for augmenting antitumor activity is applicable for any chemotherapeutic agent, some exemplary ones are irinotecan, FU, taxol, cisplatin adriamycin, oxaliplatin, cyclophasphamide, taxotere, and EGF and VGF inhibitors. In addition, the present invention can also be used for augmenting the antitumor activity of other anticancer therapies such as radiation treatment.

To demonstrate the effect of selenium in reducing the toxic effect of chemotherapeutic agents, studies were carried out in tumor bearing nude mice. It should be noted that while previous studies have reported an effect of selenium on reducing toxicity (such as cardiotoxicity) of some anticancer agents in vitro, such studies do not permit an assessment of the effect of selenium on the efficacy of anticancer agents.

In one embodiment of the present invention, it was determined that methylselenocysteine (MSC) and seleno-L-methionine (SLM) are effective agents in augmenting the antitumor activity of anticancer agents. Agents representing five different classes of clinically approved compounds were selected. Thus, the chemotherapeutic agents tested were irinotecan (topoisomerase I inhibitor); doxorubicin, (topoisomerase II inhibitor), FU (DNA synthetic inhibitor); taxol and taxotere (microtubule inhibitor) and cisplatin and oxaliplatin (DNA alkylating agents). The two selenium containing compounds were evaluated in xenograft tumors in mice for the various chemotherapeutic agents. The in vivo effects were observed using non-toxic doses of the selenium containing compounds (about 0.2 mg/mouse/day or lower).

It should also be noted that selenium containing compounds, 5-methylselenocysteine (MSC) and seleno-L-methionine (SLM) were found not to be toxic when 0.2 mg/mouse/day for 28 days was administered orally to normal nude mice and are effective modulators of toxicity induced by anticancer drugs. In one embodiment, it is demonstrated that Selenium containing compounds, MSC and SLM potentiate the cure rate of irinotecan in xenografts bearing drug sensitive and relatively resistant tumors. Further, MSC potentiates the antitumor activity of taxol, cisplatin (CDDP), oxaliplatin, cyclophosphamide, taxotere and doxorubicin (Dox) of xenografts bearing human A253 and FaDu squamous cell carcinoma of the head and neck tumors. While not intending to be bound by any particular theory, it is considered that potentiation of the efficacy of anticancer drugs is associated with increased antitumor activity and decreased toxicity.

The present invention can be used for treatment of tumors including, but not limited to, adenocarcinomas, melanomas, lymphomas, sarcomas, leukemias, and different organ tumors like lung, breast, ovarian, head and/or neck, prostate, cervical, endometrial, colorectal, gastric, liver, fallopian tubes, esophagus, small intestine, pancreas, kidney, adrenal, vaginal, vulvar, brain and testicular tumors. The combination regimen of an antitumor agent and selenium may be used with other anticancer therapies such as radiation, surgery and immunotherapy. This invention can be used for achieving antitumor effect in mammals including humans, mice, rats, dogs etc.

The following examples are provided below to illustrate the present invention. These examples are intended to be illustrative and are not to be construed as limiting in any way.

### EXAMPLE 1

This example describes the administration schedules for the selenium compounds and the anticancer agents used in Examples 2-7. In addition, the tumor xenografts established are also described.

5-Methylselenocysteine (MSC). Two schedules were evaluated: 1) in combination with irinotecan, MSC (0.2 mg/mouse/d×28) was administered orally for 28 days with the first dose administered daily for seven days prior to the weekly drug administration and 2) in combination with other drugs, MSC was administered orally (0.2 mg/mouse/d×14) daily for 14 days with the first dose administered daily for seven days prior to the single i.v. administration of taxol, CDDP, Dox, taxotere and cyclophosphamide.

Anticancer drug administration. The anticancer drugs administration schedule was as follows.
i. Irinotecan (CPT-11), weekly i.v. push for four (4) weeks,
ii. Taxol, single i.v. push,
iii. Cisplatin (CDDP), single i.v. push.
iv. Doxorubin (Dox), single i.v. push.
v. Cyclophosphamide, single i.v. push.
vi. Oxaliplatin, single i.v. push
vii. Taxotere, single i.v. push

Tumor Xenografts. The tumor xenografts (all tumors have a doubling time of approximately 3 days) were initially established by implanting subcutaneously 10⁶ cultured cells and passed several generations by transplanting 50 mg or more non-necrotic tumor tissues before treatment. The following tumor xenografts were established.
i. HCT-8: poorly-differentiated colon carcinoma, expressing wild type p53
ii. HT-29: well-differentiated colon carcinoma expressing mutant p53
iii. A253: well-differentiated squamous cell carcinoma of the head and neck (SCCHN) expressing no p53
iv. FaDu: poorly-differentiated squamous cell carcinoma of the head and neck (SCCHN) expressing mutant p53

### EXAMPLE 2

Evaluation of the Effect of Selenium Containing Compounds on the Antitumor Activity of Irinotecan.

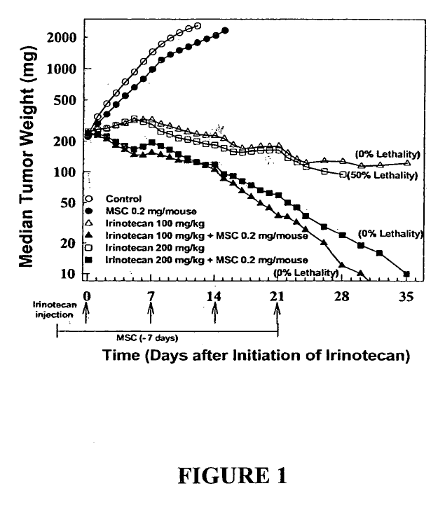

In this example, the effect of selenium on the antitumor activity of irinotecan was determined. Irinotecan was administered at 100 mg/kg/wk for 4 weeks (MTD) and 200 mg/kg/wk for 4 weeks (toxic) alone and in combination with 0.2 mg/mouse/d of MSC for 28 days to nude mice bearing HCT-8 colon xenografts. The results are shown in FIG. 1. The data indicate that although the kinetics of response to 100 mg/kg and 200 mg/kg of irinotecan in combination with MSC is similar with complete tumor regression achieved within one to two weeks after termination of treatment, MSC offered complete protection against lethal doses of irinotecan (200 mg/kg). All the animals survived treatment with irinotecan in combination with MSC compared with 50% survival of animals treated with irinotecan alone. Thus, MSC potentiates the efficacy of irinotecan by increasing cure rate and by decreasing toxicity.

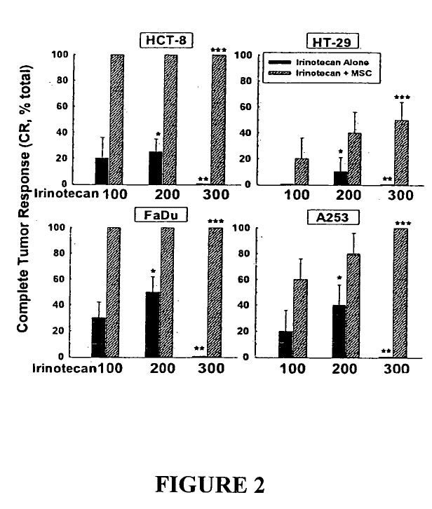

The data in FIG. 2 is the summary of cures of xenografts treated with different doses of irinotecan±MSC in two colon carcinomas (HCT-8 and HT-29) and squamous cell carcinoma of the head and neck (FaDu and A253) xenograft tumors. The maximum tolerated weekly dose of irinotecan is 100 mg/kg/wk for 4 weeks. The 200 mg/kg and 300 mg/kg are lethal doses where 50% and 100% of animals did not survive the four weeks of therapy, respectively. With the 100 mg/kg/wk for 4 weeks irinotecan (MTD), the cure rate was increased from 20% to 100% in HCT-8, from 0% to 20% in HT-29, from 30% to 100% in FaDu and from 20% to 60% in A253 xenografts. The data in FIG. 2 also indicate that while HT-29 (colon) and A253 (SCCHN) tumors are less sensitive to the MTD of irinotecan, than HCT-8 and FaDu tumors, administration of higher doses of irinotecan yield higher cure rates with 200 and 300 mg/kg/wk for 4 weeks to 40% and 50%, respectively in HT-29 and to 80% and 100% in A253 tumors, respectively. While the increase in cures by MSC with 200 mg/kg irinotecan was achieved without toxicity (lethality), increased cure by MSC with 300 mg/kg irinotecan was associated with 20% lethality. In contrast, 200 and 300 mg/kg irinotecan was associated with 50% and 100% lethality. The data in FIG. 2 demonstrates further that MSC effectively modulates the cure rate of irinotecan in several human xenograft tumors with differential response to the MTD of irinotecan.

### EXAMPLE 3

Comparative Antitumor Activity of MSC and SLM in Combination with the Maximum Tolerated Dose of Irinotecan

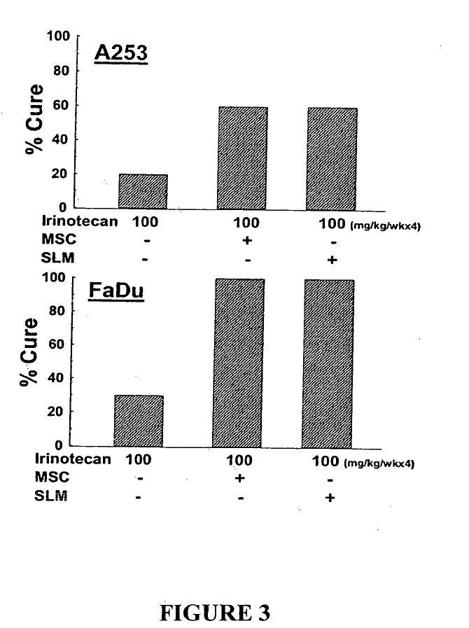

This example demonstrates that any selenium compound can be used to enhance the effects of the antitumor agents. As an illustration, MSC and SLM were used in combination with irinotecan. The results, shown in FIG. 3, represent a comparative evaluation of the effect of MSC and SLM (0.2 mg/mouse/d×28) on the antitumor activity of irinotecan (100 mg/kg/wk×4). In both A253 and FaDu, MSC and SLM produced similar potentiation of the antitumor activity of irinotecan, indicating that the effect is not specific for MSC.

### EXAMPLE 4

Comparative Evaluation of MSC and SLM as Selective Modulator of the Antitumor Activity and Toxicity of Irinotecan Administered at Twice the Maximum Tolerated Dose.

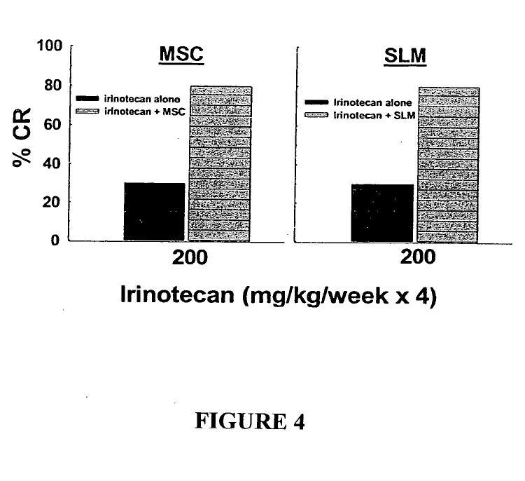

This example demonstrates that since the selenium compounds reduce the toxicity of the antitumor agents, the dose of the antitumor agents that can be administered can be increased. To determine whether therapeutic synergy achieved with irinotecan in xenografts is specific for MSC, the antitumor activity of irinotecan with and without two selenium-containing compounds, MSC and SLM were compared in xenografts bearing A253 (SCCHN) tumors for the maximum tolerated dose (200 mg/kg/wk×4). The results are presented in FIG. 4. The data compare the antitumor activity of MSC with SLM in combination with irinotecan (200 mg/kg/wk×4). The results outlined in FIG. 4 indicate that MSC and SLM produced equal potentiation of the antitumor activity of irinotecan with 80% of the treated animals were cured of their disease with no toxicity, significant lethality was observed in approximately 50% of the animals treated with this dose of irinotecan. Of the 50% of animals who survived treatment with irinotecan (200 mg/kg/wk×4) in combination with MSC or SLM 80% were cured compared with 40% cures of animals treated with irinotecan alone. Thus, MSC and SLM are equally effective in selective modulation of antitumor activity of irinotecan.

A summary of the effect of MSC in potentiation of the antitumor activity of irinotecan is presented in Table 1.

TABLE 1
5-Methylselenocysteine (MSC, 0.2 mg/mouse/d × 28) increase the cure
rate (CR) without toxicity of xenografts treated with Irinotecan

| Irino- | MSC | | | | | |
|---|---|---|---|---|---|---|
| tecan | (.02 mg/ | Sur- | | | | |
| mg/kg/ | mouse/ | vivor | % CR of Surviving Animals | | | |
| wk × 4 | d × 28) | (%) | HCT-8 | HT-29 | A253 | FaDu |
| 100 | − | 100 | 20 | 0 | 20 | 30 |
| 100 | + | 100 | 100 | 20 | 60 | 100 |
| 200 | − | 50 | 30 | 10 | 40 | 50 |
| 200 | + | 100 | 100 | 10 | 80 | 100 |
| 300 | − | 0 | †NE | NE | NE | NE |
| | | | (lethal) | (lethal) | (lethal) | (lethal) |
| 300 | + | 80 | 100 | 50 | 100 | 100 |

†NE, were not evaluable since 100% of the animals did not survive the 4 weeks of treatment.

The data shows the antitumor activity (cures) of irinotecan alone and in combination with MSC against xenografts bearing human tumors. In all four tumors, MSC potentiates significantly the antitumor activity of irinotecan. Since it was not possible to assess accurately tumor response to toxic dose of irinotecan (200 and 300 mg/kg/wk×4) due to lethality, demonstrated ability of MSC to protect normal tissues against toxic doses of irinotecan provided the opportunity for delivering the higher doses of irinotecan resulting in increased cure rates in the four human tumor xenografts evaluated. While the 300 mg/kg/wk×4 administration of irinotecan resulted in 100% lethality, in combination with MSC, % lethality was reduced to 20%.

### EXAMPLE 5

Modulation of the Antitumor Activity of Anticancer Drugs by MSC in Mice Bearing Human Tumors.

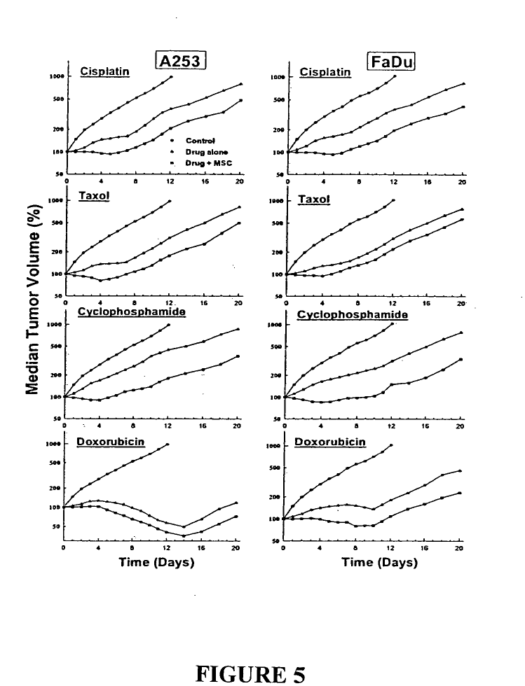

This embodiment demonstrates that selenium compounds can be used to potentiate the antitumor activity of a broad spectrum of antitumor agents. To determine whether modulation of the therapeutic efficacy and cure of irinotecan by MSC is drug specific, the antitumor activity of drugs, representing different classes and mechanisms of action were evaluated alone and in combination with non-toxic doses and schedules of MSC (0.2 mg/mouse/d×14) in xenografts bearing human A253 and FaDu (SCCHN) tumors (FIG. 5). The data in FIG. 5 represent the use of the MTD doses of cisplatin (8 mg/kg×1), cyclophosphamide (100 mg/kg×1), taxol (35 mg/kg×1) and doxorubicin (10 mg/kg×1). The results indicate that MSC potentiates the antitumor activity of each drug in xenografts bearing A253 and FaDu tumors. Potentiation of the antitumor activity by MSC was not associated with any increased toxicity with these clinically important chemotherapeutic agents. The data in FIG. 5 clearly demonstrates that MSC modulation of the antitumor activity of anticancer drugs covers a broad spectrum of anticancer agents.

### EXAMPLE 6

Effect of Selenium on the Antitumor Activity of Oxaliplatin in Rats Bearing Advanced Ward Cancer and in Nude Mice Bearing Human Squamous Cell Carcinoma for the Head and Neck A253 and FaDu Xenografts.

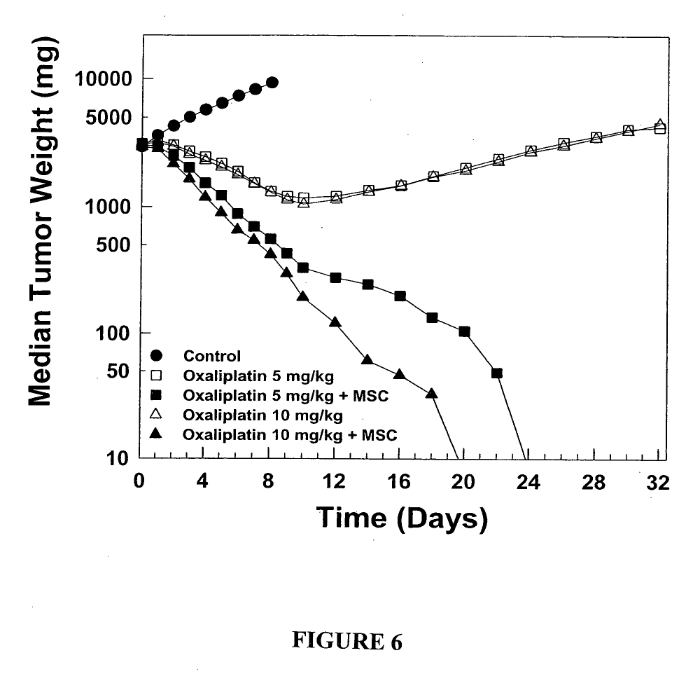

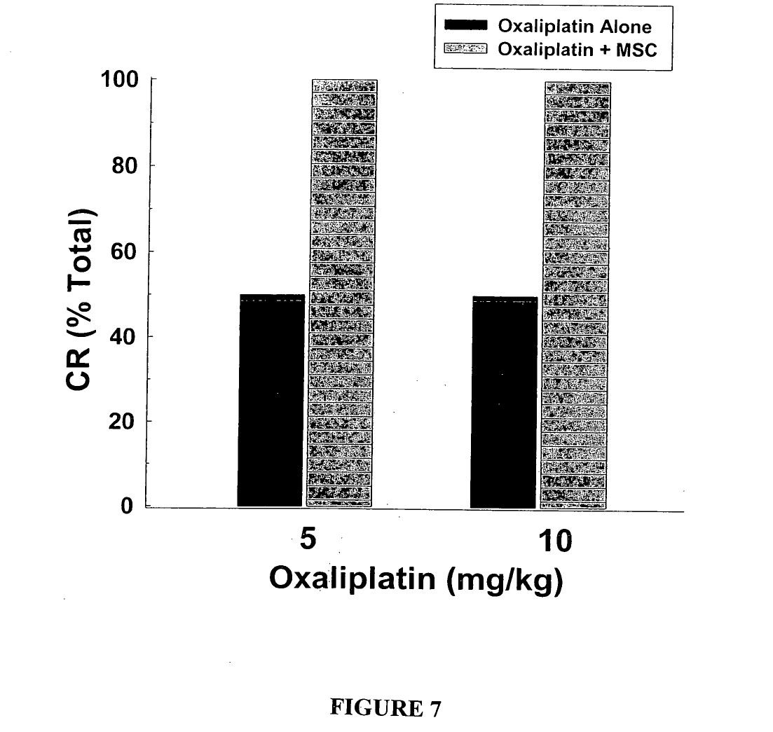

This example further demonstrates that selenium enhances the antitumor activity of a wide spectrum of antitumor agents. In this example, the effect of selenium on the antitumor activity of oxaliplatin and doxorubicin was tested as follows. The effect of selenium was determined on the antitumor activity of oxaliplatin in rats bearing advanced ward colorectal carcinoma (3000 mg). Rats received MSC (0.75 mg/rat/d) or saline fourteen days prior to single i.v. injection of oxaliplatin of 5 and 10 mg/kg with continuous daily oral administration of MSC for additional seven days for a total of 21 days of MSC and oxaliplatin is administered on day 14 after saline and MSC treatment. The data indicate that while oxaliplatin at 5 and 10 mg/kg exhibited similar antitumor activity (tumor growth inhibition), rats treated with the combination of oxaliplatin and MSC, however, demonstrated significant enhancement of tumor growth inhibition since all of the animals were without detectable tumor (cures) on about days 20-24 (FIG. 6). Of interest, optimal cure rate was only detected at approximately three weeks after the i.v. administration of a single dose of oxaliplatin (delayed antitumor effect). Further, lack of dose response with oxaliplatin is clearly evident since 5 (MTD) and 10 mg/kg yielded similar cure rate (FIG. 7). In addition, while 10 mg/kg Oxaliplatin was toxic, the observed high cure rate of oxaliplatin with MSC was without any detectable toxicity (weight loss and diarrhea). Thus, MSC is highly selective and in therapeutic trials synergistic when combined with oxaliplatin in this rat ward colorectal tumor.

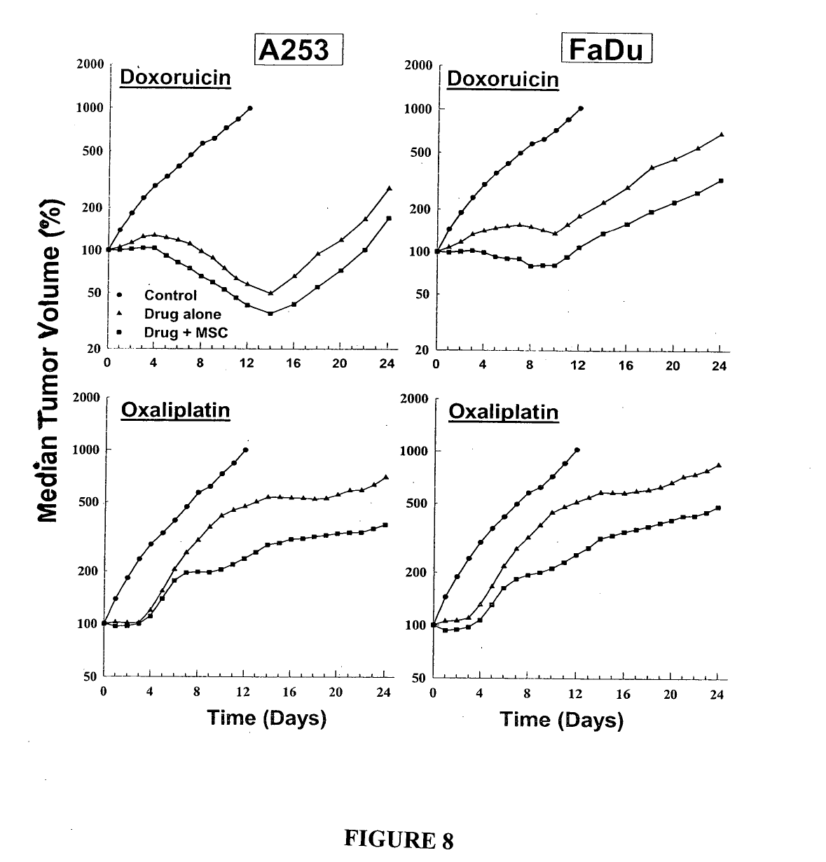

In another illustration of this embodiment, the effect of selenium on the antitumor activity of oxaliplatin and doxorubicin was tested on human squamous cell carcinoma xenografts. The data in FIG. 8 is a graphic representation of the kinetics of the antitumor response of xenograft tumors (A253/FaDu) treated with doxorubicin (10 mg/kg×1) and oxaliplatin (15 mg/kg×1) alone and in combination with MSC. In mice, MSC was orally administered at 0.2 mg/mouse/d with the first daily dose administered seven days prior to single i.v. administration of drug and continuous for additional seven days for a total MSC treatment of 14 days. The results indicate that MSC potentiates the antitumor activity of both drugs in both A253 and FaDu xenografts (FIGS. 8 & 9). The observed increase with MSC in the antitumor activity of oxaliplatin and doxorubicin was not associated with any toxicity. Thus, MSC is highly selective in potentiation of the antitumor activity of oxaliplatin and doxorubicin in xenografts bearing A253 and FaDu tumors.

In summary, the maximum tolerated doses of oxaliplatin and doxorubicin alone and in combination with MSC were compared and the data indicate that the MTD of drugs is higher when combined with MSC due to MSC protection of normal tissues for drug induced toxicity. These results are summarized in Table 2.

TABLE 2

MTD (mg/kg)

| Drug | MSC | Rats | Mice |
|---|---|---|---|
| Doxorubicin | − | 6 | 10 |
| Doxorubicin | + | 9 | 12.5 |
| Oxaliplatin | − | 15 | 7.5 |
| Oxaliplatin | + | 20 | 12.5 |
| Taxol | − | 50 | 35 |
| Taxol | + | 100 | 75 |

### EXAMPLE 7

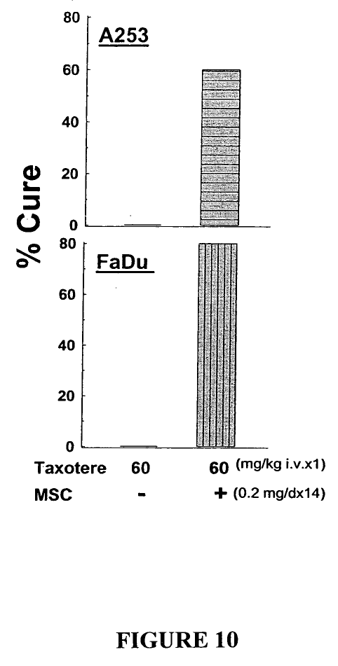

This embodiment demonstrates that selenium can augment the antitumor activity of another anticancer agent, i.e., taxotere. To illustrate this embodiment, the effect of taxotere was evaluated alone and in combination with MSC (0.2 mg/mouse/d33 14) in xenografts bearing human A253 and FaDu (SCCHN) tumors. Toxotere was administered by a single i.v. injection and MSC by p.o. daily for 14 days with the first dose started 7 days before taxotere treatment. The results (FIG. 10) show that while both tumors were insensitive to the MTD dose of taxotere (60 mg/kg), the combination of MSC and taxotere increased the number of animals cured of their tumors to 60% in A253 xenografts and to 80% in FaDu bearing xenografts. These results indicate that MSC potentiates the antitumor activity of taxotere and is capable of reversal of resistance of these tumors to taxotere.

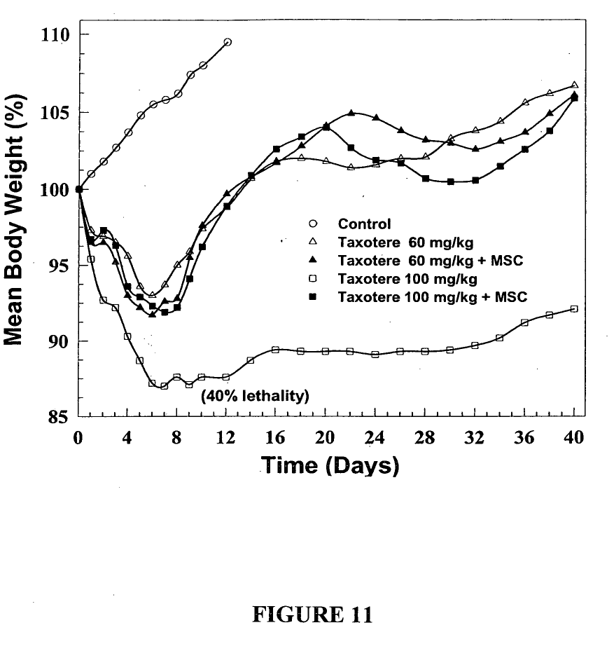

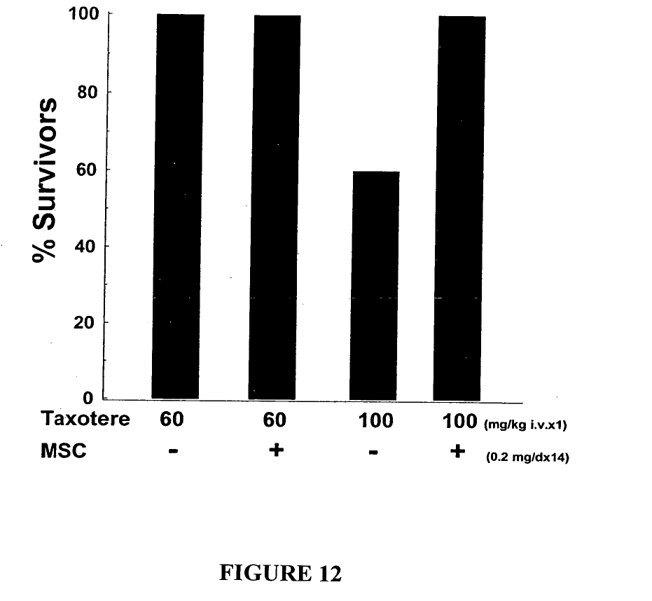

Furthermore, it was also observed that selenium protects against taxotere induced toxicity. Taxotere was administered to nude mice at non-toxic (60 mg/kg) or toxic (100 mg/kg) dose by a single i.v. injection and MSC was administered by oral route daily for 14 days with the first dose of MSC started before taxotere treatment. The results on mean body weight are shown in FIG. 11. While 100 mg/kg taxotere resulted in approximately 15% loss of total body weight, in combination with MSC, the weight loss was insignificant and similar to untreated animals. The effect on survival is shown in FIG. 12. Again, while 100 mg/kg taxotere resulted in 40% lethality, in combination with MSC, 100% of the animals treated with 100 mg/kg taxotere survived with no signs of toxicity. (FIG. 12).

These results clearly indicate that selenium compounds augment the antitumor activity of anticancer agents. The selenium compounds also reduce the toxicity of the antitumor agents and therefore, the maximum tolerated doses of the anticancer drugs is increased.

Those skilled in the art will recognize that based upon the disclosure herein, minor modifications will be apparent to those skilled in the art. Such modifications are intended to be within the scope of this invention.

1. A method for enhancing the antitumor activity of an anticancer agent selected from the group consisting of 5-fluorouracil, cyclophosphamide, taxol, irinotecan, oxaliplatin, taxotere and doxorubicin comprising the steps of administering to an individual having a tumor, a therapeutically effective dose of the anticancer agent and a selenium compound wherein the antitumor activity of the anticancer agent is greater in the presence of the selenium compound than in the absence.

2. The method of claim 1, wherein the anticancer agent is 5-fluorouracil.

3. The method of claim 1, wherein the anticancer agent is cyclophosphamide.

4. The method of claim 1, wherein the anticancer agent is taxol.

5. The method of claim 1, wherein the anticancer agent is irinotecan.

6. The method of claim 1, wherein the anticancer agent is oxaliplatin.

7. The method of claim 1, wherein the anticancer agent is doxorubicin.

8. The method of claim 1, wherein the anticancer agent is taxotere.

9. The method of claim 1, wherein the selenium compound is seleno-L-methionine.

10. The method of claim 1, wherein the selenium compound is methylselenocysteine.

11. The method of claim 1, wherein the selenium compound is administered at a time selected from the group consisting of prior to administration of the anticancer agent, during administration of the anticancer agent, following administration of the anticancer agent and a combination thereof.

12. The method of claim 1, wherein the tumor is selected from the group consisting of adenocarcinomas, melanomas, lymphomas, sarcomas, lung, breast, ovarian, head, neck, prostate, cervical, endometrial, colorectal, gastric, liver, fallopian tubes, esophagus, small intestine, pancreas, kidney, adrenal, vaginal, vulvar, brain and testes.

13. The method of claim 1, wherein the individual is also provided a treatment selected from the group consisting of surgery, radiation and immunotherapy.

14. The method of claim 1, wherein the individual is a human.

15. The method of claim 1, wherein the individual in a mouse or rat.

16. A method for using taxotere at a higher than therapeutic dose comprising the steps of administering to an individual in need of treatment a higher than therapeutic dose of taxotere and a selenium compound, wherein the toxicity of taxotere is reduced and the antitumor activity is increased with the administration of the selenium compound.

17. The method of claim 16, wherein the selenium compound is seleno-L-methionine.

18. The method of claim 16, wherein the selenium compound is methylselenocysteine.

19. The method of claim 16, wherein the selenium compound is administered at a time selected from the group consisting of prior to administration of taxotere, during administration of taxotere, following administration of taxotere and combinations thereof.
